(19) United States
(12) Patent Application Publication
(10) Pub. No.: US 2025/0228970 A1
(43) Pub. Date: Jul. 17, 2025

(54) GENE THERAPY FOR THE TREATMENT OF COGNITIVE DISORDERS

(71) Applicant: The Regents of the University of California, Oakland, CA (US)

(72) Inventors: Mark Tuszynski, La Jolla, CA (US); Krystof Bankiewicz, San Diego, CA (US)

(21) Appl. No.: 18/854,674

(22) PCT Filed: Apr. 7, 2023
(86) PCT No.: PCT/US2023/017851

§ 371 Date: Oct. 7, 2024

Related U.S. Application Data
(60) Provisional application No. 63/328,934, filed on Apr. 8, 2022.

(51) Int. Cl. A61K 48/00; A61K 9/00; A61K 38/18; A61P 25/28; C12N 15/86

(52) U.S. Cl. CPC A61K 48/005; A61K 9/0085; A61K 38/185; A61K 48/0075; A61K 48/0083; A61P 25/28; C12N 15/86; C12N 2750/14143

(57) ABSTRACT

Methods and compositions for the treatment of cognitive disorders are provided herein.

### CROSS-REFERENCE TO RELATED APPLICATION

This application claims priority under 35 U.S.C. § 119 (e) to U.S. Provisional Application Nos. 63/328,934, filed Apr. 8, 2022, the contents of which are incorporated herein by reference in its entirety.

### STATEMENT OF GOVERNMENT SUPPORT

This invention was made with government support under AG043416, and AG010435 awarded by the National Institutes of Health. The government has certain rights in the invention.

### TECHNICAL FIELD

The present disclosure relates generally to the field of methods for accurately and safely delivering gene therapy to entorhinal/hippocampal regions to treat cognitive disorders and other diseases of the central nervous system (CNS). This disclosure describes specific parameters for targeting networks of this brain region, including accurate anatomical targets, vector concentrations and vector volumes.

### SUMMARY OF THE DISCLOSURE

One embodiment of the disclosure relates to a method for improving cognitive function in a subject in need thereof comprising, or alternatively consisting essentially of, or yet further consisting of administering to a ventromedical nucleus of the subject a polynucleotide encoding a therapeutic peptide or a brain-derived neurotrophic factor (BDNF) at a dose between about 3×10¹¹ vg/ml to about 1×10¹³ vg/ml administered at an infusion rate between about 0.001 ml/minute to about 0.015 ml/minute and an infusion volume between about 250 μl to about 750 μl per hemisphere or alternatively both hemispheres, thereby improving cognitive function in the subject. Non-limiting examples of cognitive functions include short or long term memory or various aspects of dementia. Other diseases and disorders are known in the art and described herein. Methods to identify improvement are known in the art. Improvement can be measured for each individual subject as compared to a prior timepoint or against an average measurement for a group of subjects that may, or may not be suffering from the same impairment. In one aspect, the method further comprises, or consists essentially of, or yet further consist of assaying for cognitive function before or after administration of the therapy, and optionally comparison of the test results to a base line value. The assay can include an appropriate set of biomarkers or other physical or clinical parameters.

In some embodiments, the subject suffers from a condition selected from Alzheimer's disease (AD), mild cognitive impairment, pre-symptomatic AD, frontotemporal dementia, or lewy body dementia.

In one aspect, the subject being treated is pre-symptomatic, who is cognitively intract but is at high risk of developing cognitive impairment (e.g., Alzheimer's disease based on biomarkers such as cerebrospinal fluid studies and brain positron emission tomography imaging). In one aspect, the method further comprises assaying for these biomarkers from the subject before and/or after administration of the therapy and optionally comparing the results to a base line value for the general population or the subject in particular.

In some embodiments, the subject is a mammal or a human, a simian, a rat, a mouse, an equine, a feline, a canine or a sheep.

In some embodiments, the polynucleotide further comprises an expression vector and the polynucleotide is administered in the expression vector. Non-limiting examples of such include, for example a plasmid, a liposome, a lentiviral vector, an adenoviral vector, or an adeno-associated vector (AAV). Methods to make such vectors are known in the art and briefly described herein. In some embodiments, the polynucleotide is operatively linked to regulatory nucleotides to drive expression of the polynucleotide. Non-limiting examples of such regulatory nucleotides include promoters and enhancer elements.

In some embodiments, the administering comprises, or consists essentially of, or yet further consists of convection-enhanced delivery (CED). In some embodiments, the CED further comprises an infusion catheter with a step distance between about 0.5 mm to about 2.0 mm from the infusion tip, and ranges in between.

In some embodiments, the administering is not to or excludes one or more of: substantial delivery to a select region of the brain selected from one or more of a presubiculum, a parasubiculum, a subiculum or a hippocampus. In another aspect, the administration is not to or excludes substantial delivery to a select region of the brain selected from two or more, three or more of, or all of a presubiculum, a parasubiculum, a subiculum or a hippocampus.

In some embodiments, the polynucleotide is administered at 3 or 4 infusion sites in the subject.

In some embodiments, the administration is in one or more dose, and each dose comprises at least 3×10¹¹ vg/ml. In some embodiments, administration is in one or more dose, and each dose is in an amount selected from of: between about 3×10¹¹ vg/ml to about 5×10¹¹ vg/ml, between about 4×10¹¹ vg/ml to about 6×10¹¹ vg/ml, between about 5×10¹¹ vg/ml to about 7×10¹¹ vg/ml, 6×10¹¹ vg/ml to about 8×10¹¹ vg/ml, between about 7×10¹¹ vg/ml to about 9×10¹¹ vg/ml, between about 8×10¹¹ vg/ml to about 1×10¹² vg/ml, between about 9×10¹¹ vg/ml to about 2×10¹² vg/ml, between about 1×10¹² vg/ml to about 3×10¹² vg/ml, 2×10¹² vg/ml to about 4×10¹² vg/ml, 3×10¹² vg/ml to about 5×10¹² vg/ml, 4×10¹² vg/ml to about 6×10¹² vg/ml, 5×10¹² vg/ml to about 7×10¹² vg/ml, 6×10¹² vg/ml to about 8×10¹² vg/ml, 7×10¹² vg/ml to about 9×10¹² vg/ml, or 8×10¹² vg/ml to about 1×10¹³ vg/ml. In a further aspect, provided herein are compositions comprising a dose of the polynucleotide and/or vector having the aforementioned vg/ml. The compositions can further comprise a preservative or cryoprotective agent or other agent to ease delivery. In a further aspect, the composition is lyophilized.

One embodiment of the disclosure relates to a method for delivering an expression vector to a ventromedial nucleus of a subject in need thereof, comprising, or consisting essentially of, or yet further consisting of administration of the vector by infusion of the vector comprising, or consisting essentially of, or yet further consisting of: (a) an infusion catheter with a step distance between about 0.5 mm to about 2.0 mm from the infusion tip; (b) an infusion rate between about 0.001 ml/minute to about 0.015 ml/minute; (c) an infusion volume between about 250 μl to about 750 μl per hemisphere, wherein the infusion occurs between about 3 to about 4 infusion sites; and (d) a dose between about 3×10¹¹ vg/ml to about 1×10¹³ vg/ml, and wherein the delivery avoids a presubiculum, a parasubiculum, a subiculum, or a hippocampus regions and the subject has a cognitive disorder. In one aspect, the expression vector is delivered with an infusion cannula with a step design of a distance from to be between 0.5 mm-2.0 mm from the infusion tip.

In some embodiments, the expression vector further comprises a therapeutic polynucleotide. In some embodiments, the polynucleotide encodes a protein selected from the group of: brain-derived neurotrophic factor (BDNF), palmitoyl-protein thioesterase 1 (PPT1), tripeptidyl peptidase 1, CLN6 (linclin), CLN8, cathepsin D, or MFSD8 or battenin. Other therapeutic proteins with neurological function are known in the art. In one aspect, the expression vector is delivered with an infusion cannula with a step design of a distance from to be between 0.5 mm-2.0 mm from the infusion tip.

In some embodiments, the expression vector is selected from a plasmid, a liposome, a lentiviral vector, an adenoviral vector, or an adeno-associated vector (AAV). Methods to make such vectors are known in the art and briefly described herein. In some embodiments, the polynucleotide is operatively linked to regulatory nucleotides to drive expression of the polynucleotide. Non-limiting examples of such regulatory nucleotides include promoters and enhancer elements.

In some embodiments, the administration is in one or more dose, and each dose comprises at least 3×10¹¹ vg/ml. In some embodiments, administration is in one or more dose, and each dose is in an amount selected from of: between about 3×10¹¹ vg/ml to about 5×10¹¹ vg/ml, between about 4×10¹¹ vg/ml to about 6×10¹¹ vg/ml, between about 5×10¹¹ vg/ml to about 7×10¹¹ vg/ml, 6×10¹¹ vg/ml to about 8×10¹¹ vg/ml, between about 7×10¹¹ vg/ml to about 9×10¹¹ vg/ml, between about 8×10¹¹ vg/ml to about 1×10¹² vg/ml, between about 9×10¹¹ vg/ml to about 2×10¹² vg/ml, between about 1×10¹² vg/ml to about 3×10¹² vg/ml, 2×10¹² vg/ml to about 4×10¹² vg/ml, 3×10¹² vg/ml to about 5×10¹² vg/ml, 4×10¹² vg/ml to about 6×10¹² vg/ml, 5×10¹² vg/ml to about 7×10¹² vg/ml, 6×10¹² vg/ml to about 8×10¹² vg/ml, 7×10¹² vg/ml to about 9×10¹² vg/ml, or 8×10¹² vg/ml to about 1×10¹³ vg/ml. In one aspect, the expression vector is delivered with an infusion cannula with a step design of a distance from to be between 0.5 mm-2.0 mm from the infusion tip.

In some embodiments, the subject is a mammal or a human, a simian, a rat, a mouse, an equine, a feline, a canine or a sheep.

The methods as disclosed herein can be combined with additional therapies and diagnostic methods to further enhance effectiveness and reduce toxicity.

Further provided are kits comprising the compositions for the performance of the methods as described herein that optionally comprise instructions for use.

### BRIEF DESCRIPTION OF THE DRAWINGS

FIG. 1: Schematic of entorhinal cortex and its major afferent and efferent connections with the hippocampus and the cerebral cortex. The major projections from the entothinal cortex (layers 1.1-1.1.1) are to the outer molecular layer of the dentate gyrus (DG) and CM region of the hippocampus. The CAI region projects back to the deeper layers of the entorhinal cortex. The entothinal cortex also directly projects to cortical regions that are sites of long-term memory storage [31]. Reproduced from Nagahra et al. (2018) Gene Therapy (2018) 25:104-114.

FIGS. 2A-2H: Location of entorhinal cortex (EC) in rhesus monkey with the corresponding MR images. Schematic diagrams (adopted from Paxinos et al. [34]) of entorhinal cortex across four coronas planes from −2.7 to −14.85 mm relative to bregma (FIG. 2A, FIGS. 2C-2E). The entorhinal cortex is located on the ventral and medial surface of the temporal lobe, with local landmarks that include the rhinal fissure (RF), perirhinal cortex (PR), amygdala (Am), subicular area(S), and hippocampus (Hp). T1 and T2 MRI scans (FIG. 2B, FIGS. 2F-2H) with the visible landmark of the rhinal fissure (RF) indicated on the T2 image (arrows in FIG. 2A, FIG. 2B, FIG. 2D, FIG. 2G). Reproduced from Nagahra et al. (2018) Gene Therapy (2018) 25:104-114.

FIGS. 3A-3F: Real-time MRI scans of AAV2-BDNF delivery into the entorhinal cortex (entorhinal cortex) of non-human primate (FIGS. 3A-3C) result in accurately targeted BDNF delivery (FIGS. 3D-3F). (FIG. 3A) An MR-compatible needle is present passing through the cortex and striatum (arrowhead) to reach the ventral and medial entorhinal cortex (arrow). The spread of gadoteridol in the infusion site is visible (arrow). Inset shows gadoteridol signal at higher magnification. (FIG. 3B) A matching histological section from the same animal shows the spread of BDNF by immunolabeling in the same region predicted by MR imaging. (FIG. 3C) Pattern of gadolinium spread in a different subject within the entorhinal cortex on MR, and d the matching histological section. (FIG. 3E) Vector spread on MR in a third subject, with gadolinium spread along the cortical surface (arrows) and f the matching BDNF immunolabeled section. Arrows indicate a band of BDNF-containing, layer II entorhinal cells. Hp hippocampus. Scale bar, (FIG. 3D)=0.5 mm, (FIG. 3E, FIG. 3F)=1 mm. Reproduced from Nagahra et al. (2018) Gene Therapy (2018) 25:104-114.

FIGS. 4A-4D: AAV2-BDNF and AAV2-GFP primarily transduce neurons. At the infusion site, (FIG. 4A) BDNF immunolabeling and (FIG. 4B) GFP immunolabeling are predominantly observed in neurons labeled with (FIG. 4C) NeuN. (FIG. 4D) Overlay. On quantification, 88.2±3.8% of GFP-expressing cells co-label for NeuN. Scale bar=50 pm. Reproduced from Nagahra et al. (2018) Gene Therapy (2018) 25:104-114.

FIGS. 5A-5E: Distribution of BDNF-labeled neurons in entorhinal infusion sites, (FIG. 5A) 13 DNF immunolabeling in an AAV2-BDNF infusion site in the entorhinal cortex. (FIG. 5B) Map of individual cells immunolabeled for BDNF, and (FIG. 5C) zone of BDNF-containing cells used to quantify the volume of vector distribution. (FIG. 5D), (FIG. 5E) The volume of AAV2-BDNF vector infused significantly correlates with the volume of tissue containing BDNF-labeled neurons (p<0.001) and with the number of BDNF-labeled neurons (p<0.001). Reproduced from Nagahra et al. (2018) Gene Therapy (2018) 25:104-114.

FIGS. 6A-6D: BDNF spread to hippocampus. (FIG. 6A) BDNF immunoreactivity in the hippocampus of a control subject shows endogenous expression of BDNF in the mossy fiber terminal fields of the CA3 lucidum and hilus region. (FIG. 6B) Following AAV2-BDNF infusion into entorhinal cortex, BDNF immunoreactivity is visible in the hippocampal outer molecular layers (arrowheads). (FIG. 6C) Fluorescent labeling illustrates BDNF immunoreactive fibers in the outer molecular layer (OML) and not in the inner molecular layer (IML) or granule cell layer (GC) of hippo-campus. (FIG. 6D) Scatterplot showed that even smaller volumes of AAV2-BDNF infused into the entorhinal cortex can lead to a widespread (˜70%) increase of BDNF expression in the 01411. of dentate gyrus. Scale bar: (FIG. 6A, FIG. 6B) 1 mm; (FIG. 6C) 200 μm. Reproduced from Nagahra et al. (2018) Gene Therapy (2018) 25:104-114.

FIGS. 7A-7C: Shows the critical need to target entorhinal subregions to effectively and safely treat memory disorders. These figures show that the higher vector titer of 1×10¹² vg/ml-1×10¹³ vg/ml from primate Study 1004 (see Table 2) and shows that this dose is tolerated when accurately targeted to the entorhinal cortex These injections at titers up to 10¹³ vg/ml resulted in good entorhinal expression without toxicity (seizures) associated with higher doses or vector mistargeting. Arrowheads below indicate regions of BDNF gene expression after vector infusion. (FIG. 7A) Monkey 21684. (FIG. 7B) Monkey 22241. (FIG. 7C) Monkey 24647.

FIGS. 8A-8E: This figure shows that in contrast, the following infusions, limited to the entorhinal cortex only and avoiding surrounding structures result in safe vector administration without seizures. (FIG. 8A) Monkey 21011. (FIG. 8B) Monkey 25722. (FIG. 8C) Monkey 23762. (FIG. 8D) Monkey 26169. (FIG. 8E) Monkey 22295.

FIGS. 9A-9D: Examples of accurate vector targeting in four different monkeys from study 1004; these monkeys did not develop seizures. (FIG. 9A) Monkey 21684, 24 months after gene delivery. Arrows indicate region of entorhinal cortex (EC) transduction. (FIG. 9B) Monkey 24647, 24 months after gene delivery. (FIG. 9C) Monkey 22241, 24 months after gene delivery. (FIG. 9D) Monkey 26169, 2.4 months after gene delivery. Scale bars A 2 mm, B 1.6 mm, C 1.8 mm, D 2.6 mm.

FIGS. 10A-10B: Design of a new infusion cannula with a step design specially adapted for the enothrinal cortex to accurately and safely treat memory disorders. The optimal distance is between about 0.5 mm to about 2.0 mm from the infusion tip. (FIG. 10A) prior art design. (FIG. 10B) prior art design.

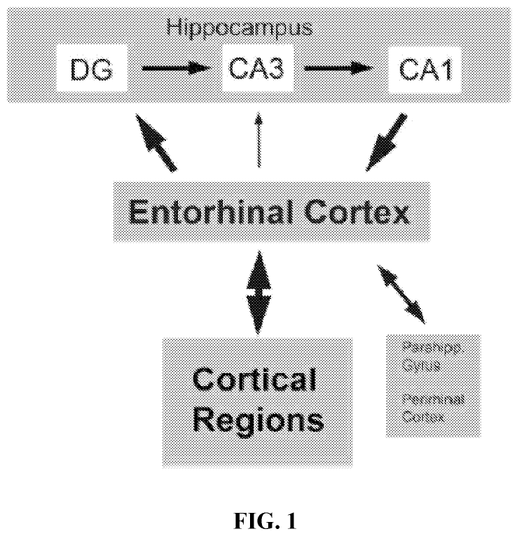

### DETAILED DESCRIPTION OF THE DISCLOSURE

### Definitions

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, or alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells and 293T cells.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal sequence identity while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity across the length of the reference sequence and exhibits substantially equivalent biological activity to the reference protein, polypeptide or nucleic acid. Alternatively, when referring to polynucleotides, an equivalent thereof is in one aspect, a polynucleotide that hybridizes under stringent conditions to the reference polynucleotide or its complement that in a further aspect, has the same or similar activity or function as the reference polynucleotide or its complement.

An equivalent of a protein or a polypeptide (referred to herein as the reference) shares at least 50% (or at least 60%, or at least 70%, or at least 80%, or at least 90%) identity to the reference and retains the reference's function and manufacturability.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter-none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can determined by incorporating them into clustalW (available at the web address: genome.jp/tools/clustalw/, last accessed on Jan. 13, 2017).

Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise, or alternatively consist essentially of, or yet further consist of two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene” refers to a polynucleotide containing at least one open reading frame (ORF) that is capable of encoding a particular polypeptide or protein after being transcribed and translated. A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, or alternatively consisting essentially of, or yet further consisting of purine and pyrimidine bases or other natural, chemically or biochemically modified, non-natural, or derivatized nucleotide bases.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, or an EF1 promoter. In some embodiments, the promoter is a chicken β-actin (“CBA”) promoter.

Additional non-limiting exemplary promoters with certain target specificity are provided herein below including but not limited to CMV, EF1a, SV40, PGK1 (human or mouse), P5, Ubc, human beta actin, CAG, TRE, UAS, Ac5, Polyhedrin, CaMKIIa, Gal1, TEF1, GDS, ADH1, CaMV35S, Ubi, H1, U6, and Alpha-1-antitrypsin. Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some embodiments, the promoter is coupled to an enhancer to increase the transcription efficiency. Non-limiting examples of enhancers include an RSV enhancer or a CMV enhancer.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, the term “vector” refers to a non-chromosomal nucleic acid comprising, or alternatively consisting essentially of, or yet further consisting of an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, or alternatively consisting essentially of, or yet further consisting of DNA condensed with cationic polymers such as heterogeneous polylysine, defined-length oligopeptides, and polyethylene imine, in some cases contained in liposomes; and the use of ternary complexes comprising, or alternatively consisting essentially of, or yet further consisting of a virus and polylysine-DNA.

A “viral vector” is defined as a recombinantly produced virus or viral particle that comprises, or alternatively consists essentially of, or yet further consists of a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5 (7): 823-827.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, or alternatively consisting essentially of, or yet further consisting of the viral genome or part thereof, and a transgene. Adenoviruses (Ads) are a relatively well characterized, homogenous group of viruses, including over 50 serotypes. Ads do not require integration into the host cell genome. Recombinant Ad derived vectors, particularly those that reduce the potential for recombination and generation of wild-type virus, have also been constructed. Such vectors are commercially available from sources such as Takara Bio USA (Mountain View, CA), Vector Biolabs (Philadelphia, PA), and Creative Biogene (Shirley, NY). Wild-type AAV has high infectivity and specificity integrating into the host cell's genome. See, Wold and Toth (2013) Curr. Gene. Ther. 13 (6): 421-433, Hermonat & Muzyczka (1984) Proc. Natl. Acad. Sci. USA 81:6466-6470, and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise, or alternatively consist essentially of, or yet further consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In some embodiments, the signal peptide is removed following localization. Examples of signal peptides are well known in the art. Non-limiting examples are those described in U.S. Pat. Nos. 8,853,381, 5,958,736, and 8,795,965.

As used herein, the term “viral capsid” or “capsid” refers to the proteinaceous shell or coat of a viral particle. Capsids function to encapsidate, protect, transport, and release into host cell a viral genome. Capsids are generally comprised of oligomeric structural subunits of protein (“capsid proteins”). As used herein, the term “encapsidated” means enclosed within a viral capsid.

As used herein, the term “helper” in reference to a virus or plasmid refers to a virus or plasmid used to provide the additional components necessary for replication and packaging of a viral particle or recombinant viral particle, such as the modified AAV disclosed herein. The components encoded by a helper virus may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising, or alternatively consisting essentially of, or yet further consisting of one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises, or alternatively consists essentially of, or yet further consists of a heterologous polynucleotide (i.e. a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

In some embodiments, the AAV is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAV rh.74 genome is provided in U.S. Pat. No. 9,434,928, incorporated herein by reference. U.S. Pat. No. 9,434,928 also provide the sequences of the capsid proteins and a self-complementary genome. In one aspect, the genome is a self-complementary genome. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV (rAAV) genomes of the disclosure comprise, or alternatively consist essentially of, or yet further consist of a nucleic acid molecule encoding a therapeutic protein (e.g., BDNF) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV PHP.B and AAV rh74. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). The nucleotide sequences of the genomes of various AAV serotypes are known in the art.

A “composition” is intended to mean a combination of active polypeptide, polynucleotide or antibody and another compound or composition, or active (e.g., a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present disclosure is also useful for veterinary treatment of companion mammals, exotic animals and domesticated animals, including mammals, rodents, and the like. In one embodiment, the mammals include horses, dogs, and cats. In another embodiment of the present disclosure, the human is an adolescent or infant under the age of eighteen years of age.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. In one aspect, the term “treatment” excludes prevention or prophylaxis.

The term “suffering” as it related to the term “treatment” refers to a patient or individual who has been diagnosed with or is predisposed to a disease.

The term “condition” refers to a disorder, illness, sickness, or disease.

The term “cognitive function” refers to multiple mental abilities including but not limited to learning, thinking, reasoning, remembering, problem solving, decision making, and attention.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, in some embodiments the effective amount is the amount sufficient to result in regaining part or full function of a gene that is deficient in a subject. In other embodiments, the effective amount of an AAV viral particle is the amount sufficient to result in expression of a gene in a subject. The skilled artisan will be able to determine appropriate amounts depending on these and other factors.

In some embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation. In some embodiments, administration is convection-enhanced delivery (“CED”).

As used herein, the term “convection-enhanced delivery” or “CED” refers to a therapeutic strategy to facilitate the targeted delivery of a pharmaceutical to the brain. In some embodiments, the CED involves a minimally invasive surgical exposure of the brain and the placement of an infusion catheter. In some embodiments, the infusion catheter has a step distance between about 0.5 mm and 2.0 mm from the catheter tip. As used herein, the term “step” refers to the extension of the cannula beyond the end of the needle tip.

As used herein, the term “hemisphere” refers to two distinct cerebral hemispheres of the brain that are connected by corpus callosum.

BDNF (brain-derived neurotrophic factor) is a 27 kDa homodimer originally derived from the human brain and promotes the outgrowth of spinal sensory neurons. In some embodiments, BDNF supports the survival and outgrowth of sensory neurons, ganglion neurons, dopaminergic neurons, cholinergic neurons, GABAergic neurons, and motor neurons. GenBank M61176 sets forth the coding sequence (mRNA) for BDNF, last accessed on Apr. 7, 2022.

### Modes for Carrying Out the Disclosure

In a prior study (Nagahra et al. (2018) Gene Therapy 25:104-114), Applicant reported that brain-derived neurotrophic factor (BDNF) gene delivery to the entorhinal cortex is a candidate for treatment of Alzheimer's disease (AD) to reduce neurodegeneration that is associated with memory loss. Accurate targeting of the entorhinal cortex in AD is complex due to the deep and atrophic state of this brain region. Using MRI-guided methods with convection-enhanced delivery, Applicant accurately and consistently targeted AAV2-BDNF delivery to the entorhinal cortex of non-human primates; 86±3% of transduced cells in the targeted regions co-localized with the neuronal marker NeuN. The volume of AAV2-BDNF (3×10⁸ vg/pl) infusion linearly correlated with the number of BDNF labeled cells and the volume (mm³) of BDNF immunoreactivity in the entorhinal cortex. BDNF is normally trafficked to the hippocampus from the entorhinal cortex; Applicant also found that BDNF immunoreactivity was elevated in the hippocampus following therapeutic BDNF vector delivery to the entorhinal cortex, achieving growth factor distribution through key memory circuits.

In contrast to this earlier study, Applicant describes herein specific methods for gene therapy to the entorhinal/hippocampal system for the treatment of cognitive disorders, or as prophylaxis against impending cognitive disorders. The present methods specify a novel and previously unknown set of specific parameters that are required to treat entorhinal/hippocampal systems in humans. These methods have application in the Alzheimer's disease (AD).

Targeting the entorhinal cortex to treat human cognitive disorders requires gene delivery to a subregion of the entorhinal cortex, the ventromedial nucleus, and avoidance of the pre-subiculum, parasubiculum, subiculum and hippocampus. If these parameters are not followed, the treatment lacks efficacy and results in neural toxicity, including seizures and risk of death. To the best of Applicant's knowledge, the literature does not disclose the need for targeting this subregion of the entorhinal cortex, nor the need to avoid vector injection into the pre-subiculum, parasubiculum, subiculum and hippocampus.

Vector volumes for targeting a sufficient volume of the entorhinal cortex for effective human treatment can, in one embodiment, be in a range of 250-750 μl per hemisphere divided among 3-4 infusion sites. To the best of Applicant's knowledge, prior literature recommended a far lower vector range of only 2.5-25 μl per site and which Applicant now knows is ineffective. In other published literature, a vector dose of 15-130 μl per site (up to 375 μl per hemisphere) is recommended, but this is also incorrect because Applicant knows that it is too low a dose. Applicant's dose is proper and based upon the work described herein wherein infusion of 30-870 μl per site over 3-4 sites (from 360-2610 μl or alternatively 250-750 μl per hemisphere) has been determined to be most effective.

Applicant has also identified new vector concentrations that are effective and safe for use in humans. Applicant found that gene therapy vector doses less than 3×10¹¹ vg/ml are ineffective, and doses higher than 1×10¹³ vg/ml can be toxic. To the best of Applicant's knowledge, the literature previously cited a range of vector doses between 1×10¹⁰ vg/ml to 1×10¹⁵ vg/ml which Applicant has since determined is incorrect. The literature described only one dose, 3×10¹¹ vg/ml, and no data from a higher range that Applicant now identify as up to 1×10¹³ vg/ml. Thus, the specific parameters of vector concentrations have not been reported previously and constitute important methods for successful and safe gene delivery.

To the best of Applicant's knowledge, the literature does not identify optimal infusion rate parameters. Based on new data from animal subjects, Applicant defines in one aspect, an optimal rate of vector infusion of 1-15 μl/min to achieve adequate vector spread and coverage of the intended target. Volumes less than this result in ineffective vector distribution in the brain; volumes exceeding this cause tissue damage and associated toxicity.

To the best of Applicant's knowledge, the literature does not specify the optimal hardware for vector infusion to achieve adequate vector spread and coverage of the intended target. Applicant now identify that the needle required to achieve adequate vector distribution in the target region should have a single, widened “step” (expansion in outer diameter) located 0.5-2.0 mm from the infusion tip in order to reduce vector reflux and loss up the infusion tract.

In addition to the preceding points, Applicant now know that these methods are relevant to treating the prodromal state of Alzheimer's disease, Mild Cognitive Impairment, and for treating patients potentially at risk for developing Alzheimer's disease. Applicant have an indication that these methods can be used to treat pre-symptomatic patients, who are cognitively intract but are at high risk of developing Alzheimer's disease based on biomarkers such as cerebrospinal fluid studies and brain positron emission tomography imaging.

### Treatment Methods

Applicant discloses methods of improving cognitive function in a subject in need thereof comprising administering to a ventromedical nucleus of the subject a polynucleotide encoding brain-derived neurotrophic factor (BDNF) at a dose between about 3×10¹¹ vg/ml to about 1×10¹³ vg/ml administered at an infusion rate between about 0.001 ml/minute to about 0.015 ml/minute and an infusion volume between about 250 μl to about 750 μl per hemisphere, thereby improving cognitive function in the subject. In a further aspect, administration further comprises an infusion catheter with a step distance between about 0.5 mm to about 2.0 mm from the infusion tip.

Further disclosed herein are methods for delivering an expression vector to a ventromedial nucleus of a subject in need thereof, comprising infusion of the vector by: (a) an infusion catheter with a step distance between about 0.5 mm to about 2.0 mm from the infusion tip; (b) an infusion rate between about 0.001 ml/minute to about 0.015 ml/minute; (c) an infusion volume between about 250 μl to about 750 μl per hemisphere, wherein the infusion occurs between about 3 to about 4 infusion sites; and (d) a dose between about 3×10¹¹ vg/ml to about 1×10¹³ vg/ml, and wherein the delivery avoids a presubiculum, a parasubiculum, a subiculum, or a hippocampus regions and the subject has a cognitive disorder. In a further aspect, administration further comprises an infusion catheter with a step distance between about 0.5 mm to about 2.0 mm from the infusion tip.

In some embodiments, a subject is a mammal. In some embodiments, a subject is a human. In some embodiments, the subject is a non-human primate. In some embodiments, the subject has a condition. In some embodiments the condition is a cognitive disorder. In some embodiments, the cognitive disorder is Alzheimer's disease (AD), mild cognitive impairment, pre-symptomatic AD, frontotemporal dementia, or lewy body dementia. In some embodiments, a subject is pre-symptomatic and cognitively intact but is at high risk of developing AD based on diagnostic biomarkers to include but not limited to cerebrospinal fluid studies and brain positron emission tomography imaging.

Titers of expression vectors to be administered in methods of the disclosure will vary depending, for example, on the particular vector, the mode of administration, the treatment goal, the individual, and the cell type(s) being targeted, and may be determined by methods standard in the art. In some embodiments, any of the polynucleotides, plasmids, viral vectors, or compositions disclosed herein are administered at a dose range from at least about 3×10¹¹ vg/ml, about 4×10¹¹ vg/ml, about 5×10¹¹ vg/ml, about 6×10¹¹ vg/ml, about 7×10¹¹ vg/ml, about 8×10¹¹ vg/ml, about 9×10¹¹ vg/ml, about 1×10¹² vg/ml, about 2×10¹² vg/ml, about 3×10¹² vg/ml, about 4×10¹² vg/ml, about 5×10¹² vg/ml, about 6×10¹² vg/ml, about 7×10¹² vg/ml, about 8×10¹² vg/ml, about 9×10¹² vg/ml, to about 1×10¹³ vg/ml. In some embodiments, the dose of expression vector is between about 5×10¹¹ vg/ml, between about 4×10¹¹ vg/ml to about 6×10¹¹ vg/ml, between about 5×10¹¹ vg/ml to about 7×10¹¹ vg/ml, 6×10¹¹ vg/ml to about 8×10¹¹ vg/ml, between about 7×10¹¹ vg/ml to about 9×10¹¹ vg/ml, between about 8×10¹¹ vg/ml to about 1×10¹² vg/ml, between about 9×10¹¹ vg/ml to about 2×10¹² vg/ml, between about 1×10¹² vg/ml to about 3×10¹² vg/ml, 2×10¹² vg/ml to about 4×10¹² vg/ml, 3×10¹² vg/ml to about 5×10¹² vg/ml, 4×10¹² vg/ml to about 6×10¹² vg/ml, 5×10¹² vg/ml to about 7×10¹² vg/ml, 6×10¹² vg/ml to about 8×10¹² vg/ml, 7×10¹² vg/ml to about 9×10¹² vg/ml, or 8×10¹² vg/ml to about 1×10¹³ vg/ml. In some embodiments, the dose is at least 3×10¹¹ vg/ml. In a further aspect, administration further comprises an infusion catheter with a step distance between about 0.5 mm to about 2.0 mm from the infusion tip.

In some embodiments, any of the polynucleotides, plasmids, viral vectors, or compositions disclosed herein are administered at an infusion rate between about 0.001 ml/minute to about 0.015 ml/minute. Infusion rates may range from at least about 0.001 ml/minute, about 0.0015 ml/minute, about 0.002 ml/minute, about 0.0025 ml/minute, 0.003 ml/minute, about 0.0035 ml/minute, 0.004 ml/minute, about 0.0045 ml/minute, 0.005 ml/minute, about 0.0055 ml/minute, 0.006 ml/minute, about 0.0065 ml/minute, 0.007 ml/minute, about 0.0075 ml/minute, 0.008 ml/minute, about 0.0085 ml/minute, 0.009 ml/minute, about 0.0095 ml/minute, about 0.01 ml/minute, to about 0.015 ml/minute. In a further aspect, administration further comprises an infusion catheter with a step distance between about 0.5 mm to about 2.0 mm from the infusion tip.

In some embodiments, any of the polynucleotides, plasmids, viral vectors, or compositions disclosed herein are administered at a dose infusion volume between about 250 μl to about 750 μl per hemisphere of the brain. Infusion volume may range from at least about 250 μl, about 275 μl, about 300 μl, about 325 μl, about 350 μl, about 375 μl, about 400 μl, about 425 μl, about 450 μl, about 475 μl, about 500 μl, about 525 μl, about 550 μl, about 575 μl, about 600 μl, about 625 μl, about 650 μl, about 675 μl, about 700 μl, about 725 μl, to about 750 μl. In some embodiments, infusion volume is administered from about 3 to about 4 infusion sites. In a further aspect, administration further comprises an infusion catheter with a step distance between about 0.5 mm to about 2.0 mm from the infusion tip.

In some embodiments, any of the polynucleotides, plasmids, viral vectors, or compositions disclosed herein are administered to the subject at least 1, 2, 3, 4, 5, 6, 7, 8, 9, or 10 times a day. In some embodiments, any of the polynucleotides, plasmids, viral vectors, or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the polynucleotides, plasmids, viral vectors, or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the polynucleotides, plasmids, viral vectors, or compositions disclosed herein are administered to the subject at least every 1, 2, 3, 4, 5, 6, 7, 8, 9, or 10 days. In some embodiments, any of the polynucleotides, plasmids, viral vectors, or compositions disclosed herein are administered to the subject at least every 1, 2, 3, 4, 5, 6, 7, 8, 9, 10, 11, 12, 13, or 14 weeks. In some embodiments, any of the polynucleotides, plasmids, viral vectors, or compositions disclosed herein are administered to the subject for a period of at least 1, 2, 3, 4, 5, 6, 7, 8, 9, or 10 days. In some embodiments, any of the polynucleotides, plasmids, viral vectors, or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the polynucleotides, plasmids, viral vectors, or compositions disclosed herein are administered to the subject for a period of at least 1, 2, 3, 4, 5, 6, 7, 8, 9, 10, 12, 13, 14, 15, 16, 17, 18, 19, or 20 months. In a further aspect, administration further comprises an infusion catheter with a step distance between about 0.5 mm to about 2.0 mm from the infusion tip.

In some embodiments, the methods disclosed herein comprise administering any of the polynucleotides, plasmids, viral vectors, or compositions disclosed herein locally. In some embodiments, the methods disclosed herein comprise administering any of the polynucleotides, plasmids, viral vectors, or compositions disclosed herein to one or more tissues. In some embodiments, the tissue is selected from muscle, epithelial, connective, and nervous tissue. In some embodiments, the tissue is brain. In some embodiments, the tissue is subregion of the entorhinal cortex of the brain. In some embodiments, the subregion is the ventromedical nucleus. In some embodiments, administration avoids the presubiculum, parasubiculum, the subiculum, and the hippocampus of the brain.

In some embodiments, the methods disclosed herein comprise administering any of the polynucleotides, plasmids, viral vectors, or compositions disclosed herein, by convection-enhanced delivery (CED). In some embodiments, the CED comprises an infusion catheter with a step distance between about 0.5 mm to about 2.0 mm from the infusion tip. In some embodiments, the step distance may range from at least about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, to about 2.0 mm. In some embodiments, the step reduces reflux and loss of up the infusion track.

In some embodiments, the polynucleotide encodes brain derived neurotrophic factor (BDNF). In some embodiments, the polynucleotide further comprises an expression vector. In some embodiments, the polynucleotide is operatively linked to a regulatory nucleotide. In some embodiments, the expression vector is a lentoviral vector, an adenoviral vector, or an adeno-associated vector (AAV).

In a further aspect, administration further comprises an infusion catheter with a step distance between about 0.5 mm to about 2.0 mm from the infusion tip.

Applicant have an indication that these methods can be used to treat pre-symptomatic patients, who are cognitively intract but are at high risk of developing Alzheimer's disease based on biomarkers such as cerebrospinal fluid studies and brain positron emission tomography imaging.

As is apparent to those of skill in the art, the aforementioned methods and compositions can be combined with other therapeutic composition and agents for the treatment or the disclosed diseases or conditions.

### Methods for Producing AAV Vectors

Disclosed herein are methods of producing an adeno-associated viral (AAV) vector (e.g., virus or viral particle) and supplement methods of producing AAV vectors are known in the art. For instance, such methods are disclosed in, for example, WO 01/83692, which is incorporated by reference herein in its entirety. General principles of AAV production are reviewed in, for example, Carter, Current Opinions in Biotechnology 1533-1539, 1992; and Muzyczka, Curr. Topics in Microbial. and Immunol. 158:97-129, 1992, each of which are incorporated by reference in their entirety. Various approaches for producing AAVs are described in Ratschin et al., Mol. Cell. Biol. 4:2072, 1984; Hermonat et al., Proc. Natl. Acad. Sci. USA, 81:6466, 1984; Tratschin et al., Mol. Cell. Biol. 5:3251, 1985; McLaughlin et al., J. Virol., 62:1963, 1988; and Lebkowski et al., Mol. Cell. Biol., 7:349, 1988; Samulski et al., J. Virol., 63:3822-3828, 1989; U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-1250, 1995; Paul et al., Human Gene Therapy 4:609-615, 1993; Clark et al., Gene Therapy 3:1124-1132, 1996; U.S. Pat. Nos. 5,786,211; 5,871,982; and 6,258,595, each of which are incorporated by reference in their entirety. In some embodiments, the method for producing an adeno-associated viral (AAV) vector comprises transducing a cell with any of the AAV packaging systems disclosed herein. In some embodiments, the cell is a eukaryotic cell. In some embodiments, the cell is a mammalian cell. In some embodiments, the cell is a recombinant cell that stably expresses the adeno-associated virus rep and cap genes. In some embodiments, the method further comprises culturing the cell to produce a population of transduced cells. In some embodiments, the method further comprises collecting the supernatant from the population of transduced cells. In some embodiments, the method further comprises subjecting the supernatant to one or more purification steps to produce a purified AAV vector sample, wherein the AAV vector sample is substantially free from cellular debris and proteins. Alternatively, or additionally, the method further comprises lysing the population of transduced cells to produce a cellular lysate. In some embodiments, the method further comprises subjecting the cellular lysate to one or more purification steps to produce a purified AAV vector sample, wherein the AAV vector sample is substantially free from cellular debris and proteins. In some embodiments, the purity of the purified AAV vector sample is at least 85%, 86%, 87%, 88%, 89%, 90%, 91%, 92%, 93%, 94%, 95%, 96%, 97%, 98%, 99% or 100% pure.

The virus, e.g., AAV, can be packaged using a viral packaging system such as a retroviral, adenoviral, herpes virus, or baculovirus packaging system. In some embodiments, packaging is achieved by using a helper virus or helper plasmid and a cell line. The helper virus or helper plasmid contains elements and sequences that facilitate the delivery of genetic materials into cells. In another aspect, the helper plasmid or a polynucleotide comprising, or alternatively consisting essentially of, or yet further consisting of the helper plasmid is stably incorporated into the genome of a packaging cell line, such that the packaging cell line does not require additional transfection with a helper plasmid.

### Vector Production and Preparation

Prior art methods can be used for vector production. As disclosed in Nagahara et al. (2018), and reproduced here, AAV2-BDNF vector was produced by UNC Vector Core (University of North Carolina) The vector genome consists of the human BDNF cDNA with a CAG promoter consisting of human cytomegalovirus (CMV) enhancer, chicken 13-actin promoter and splice donor, intron, a rabbit f1-globin splice acceptor. The vector is similarly designed to AAV2 vector used in Phase 1 and 2 gene therapy trials [Tuszynski et al., JAMA Neurol. 2015; Arvanitakis et al., AbstrAm Acad Neurol. 2007; Rafii et al., Atzheimers Demerit. 2014.]. Briefly, vector particles were prepared by transient transfection of plasmid DNA into 293 cells and purified by C₅Cl₂ centrifugation, FPLC, and sterile filtration. AAV2-BDNF vector was delivered at a titer of 3×10¹² vg/ml and aliquoted into 100 μl volumes. For infusions, AAV2-BDNF was infused at a concentration of 3×10¹¹ vg/ml. The MR contrast agent gadoteridol (Prohance, Bracco Diagnostic, Princeton, NJ) was mixed into the vector at a final concentration of 1 mM. A subset of subjects (N=9) also received AAV2-GFP at 0.03×10¹² vg/ml titer to confirm the spread of the viral vector in the transduction of cells.

### Surgical Exposure

Prior art methods with Applicant's disclosed modifications can be used for surgical methods. As disclosed in Nagahara et al. (2018), and reproduced herein, after induction of anesthesia, the animal's head was placed in a stereotactic frame and flexed in a prone position. The head was shaved and cleaned using Nolvasan solution and alcohol. A sterile field was created and a midline incision performed through the skin, muscle, and fascia by electro-cautery (Surgistat Electrosurgery, Valleylab Inc., Boulder, CO). Gentle retraction of fascia and muscle allowed for cranial exposure over cortical entry sites. Small burr holes were performed according to stereotactic coordinates to expose the dura over each of the intended infusion sites. The infusion system included CO a “ball-joint” style array (Hayes Manufacturing Services Inc., USA) that was secured to the skull by titanium screws over the craniotomies, and (ii) a custom-designed cannula (Richardson et al. [Richardson et al., Mol Ther. 2011; Richardson et al., Stereotact Fund Neurosurg. 39. 2011.]). The custom-designed, ceramic, fused silica reflux-resistant cannula consists of an 8-25-cm ceramic section (1.68 mm outer diameter (OD)) in the main portion of the shaft, and an 18-mm fused silica section that tapers down to 0.7 mm OD. The final 3-mm section is a fine fused silica lip (0.36 mm OD). Briefly, the cannula is connected to a loading line containing the infusate, and flow is regulated with a 1-ml syringe mounted onto an MRI-compatible infusion pump. The target is selected and optimal trajectory is established using neuronavigation software on baseline MRI images of the animal. Then, distance from the target to the top of the guide-stem is determined in silica, and a depth-stop is secured at the insertion distance in the cannula. After the optimal infusion parameters are determined, the cannula is manually inserted through the guiding-stem of the array to the target.

### MRI-Guided Infusion Procedure

In the prior art methods (Nagahara et al. (2018), that can be modified as disclosed herein, Applicant used a 3-Tesla Siemens Magnetom Avanto scanner (Siemens Medical Solutions, Erlangen, Germany) with Siemens resident software. Vector infusion procedures were done using three types of scanning protocols that were varied throughout the gene delivery procedure: a T1 protocol to optimize the visualization of brain structures for the purpose of needle targeting, and modified T2 protocol to obtain a rapid scan to assess vector spread, and an MP-RAGE protocol to optimize the visualization of white matter structure and vector spread (repetition time: 2110 ms; echo time: 3.6 ms; flip angle: 15°; number of excitations: 1 (repeated three times); matrix: 240×240; field of view: 240×240×240).

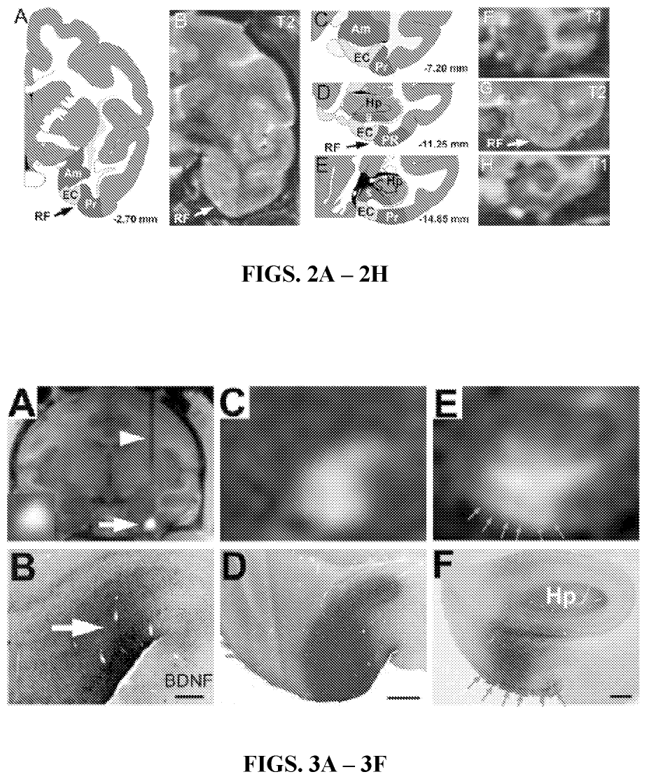

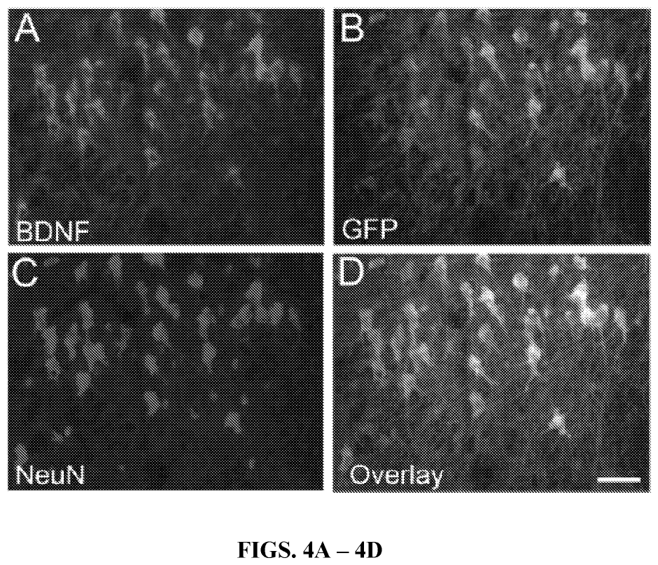

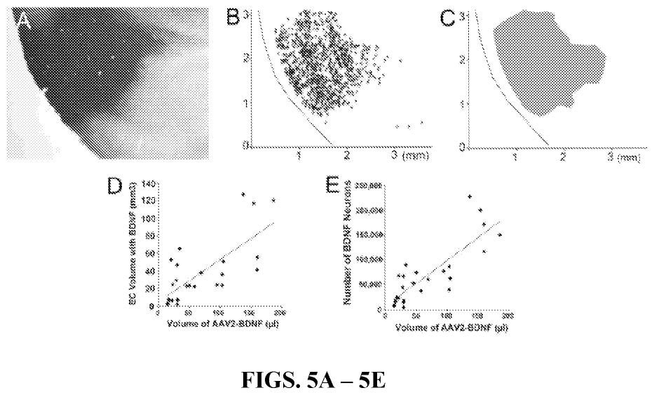

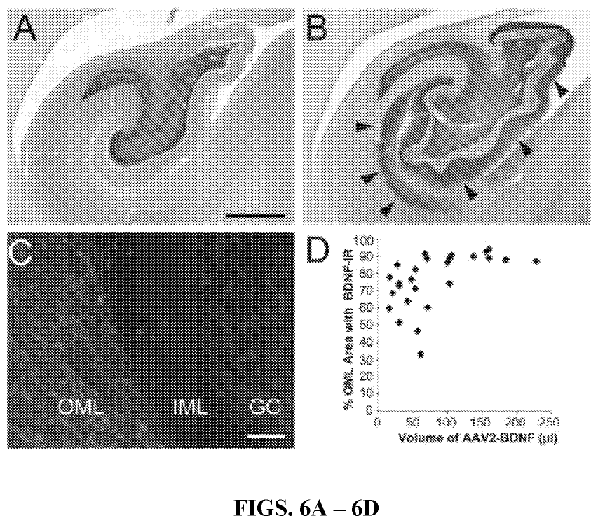

The animals were sedated with an intramuscular injection of ketamine (10 mg/kg IM) and medetomidine (0.015 mg/kg IM), intubated, and a venous line established with a 22-24-gauge catheter positioned in the cephalic or saphenous vein to deliver isotonic fluids at a rate of 5-10 ml/kg/h. Isoflurane inhalation anesthesia (Aerrane, Omeda PPD Inc., Liberty, NI) was delivered at 1-3% to maintain a stable plane of anesthesia. Then, after placement into an MRI-compatible stereotaxic frame in a supine position, two burr holes were placed for implantation of ball-joint arrays that consist of a base (attached to the skull) and a guide tube array (cylinder with three holes) [Salegio et al., Methods Mol Biol. 2016.]. The monkeys were then transferred to the MRI scanner suite. The infusion needle consisted of a ceramic silica reflux-resistant cannula with a 1-mm step at the distal end to prevent vector reflux up the cannula (MRI Intervention, Irvine). [San Sebastian et al., Mol Ther Methods Clin Dev. 2014.]. Then a 36-inch high-pressure intravenous tubing connected the infusion needle to a 1-ml syringe pump (Medfusion 3500 syringe pump, Medfusion, St Paul, MN). Prior to loading into the infusion system, the vector was adjusted to a final concentration of 3.0×10¹¹ vg/ml. In nine subjects, one-tenth of the vector infusate volume consisted of AAV2-GFP (3×10¹¹ vg/ml) and 90% of the vector consisted of AAV2-B DNF (3×10¹¹ vg/ml); these monkeys enabled analysis of the type of cells in the brain that were transduced by the virus. In initial subjects, the infusion needle was placed into the skull-based stereotaxic frame and advanced into the brain to a point calculated to be located in the mid-striatum; the accuracy of the needle trajectory and depth were then confirmed with a T2 scan. Because none of these mid-trajectory scans required correction of needle trajectory, all subsequent subjects underwent lowering of the infusion needle into the entorhinal cortex itself, stopping at a point calculated on initial MRI scans to be located 1 mm dorsal to the intended target region. Then, a T1, MP-RAGE and T2 weighted set of images were obtained. Based on these images, applicant then advanced the infusion needle to the distance required to reach a point within the entorhinal cortex that was located 1 mm from the ventral brain surface (FIG. 2).

The monkeys received 1-3 infusion sites into the entorhinal cortex per side of the brain. For a single infusion per side, the injection was placed approximately at the midpoint of the antero-posterior length of the hippocampus. For entorhinal cortex that received 2-3 infusions, attempts were made to spread the vector through the majority of the volume of the entorhinal cortex. For this study, 34 entorhinal cortices were infused with AAV2-BDNF (3×10¹¹ vg/mL): 18 received a single infusion site per side of the brain, nine received two infusions, and seven receiving three infusions.

At the point that infusion needles were going to penetrate the brain, infusions pumps were turned on at a rate of 3 μU min to maintain positive pressure and prevent needle blockade as the needle was advanced through the brain to the target. Once the final target was confirmed on MRI, the infusion rate was adjusted ramping from 1 up to 3 pl/min to achieve vector spread through the entorhinal cortex region located in the 1-mm-thick MRI slice containing the infusion needle. Rather than infuse a pre-set volume of vector, applicant continued infusions until the target region of interest (about 5 mm of the rostral-cadual extent of the entorhinal cortex) was covered by gadoteridol. Scans were continuously obtained through the infusion procedure, as previously described [Su et al., Mot Ther. 2010; San Sebastian et al. Mol Ther Methods Clin Dev. 2014.]. The range of vector volume infused per site was 15-160111. Vital signs were monitored continuously during the surgery.

After completion of infusions, the subjects were returned to the surgical suite for removal of the skull-based stereotaxic frame. Then, the animals received an intramuscular injection of NSAID (Meloxicam) and buprenorphine (Buprenex) the day after the convection-enhanced delivery (CED) infusion as part of the post-procedural analgesia management. Once the animal returned to its cage, it was evaluated twice daily for 5 days by veterinary staff. Detailed, standardized forms were completed for each animal that included evaluations of the surgical-site integrity, edema, infection, balance, locomotion, attitude, food intake, and fecal and urine output. No abnormalities or signs of discomfort were reported.

### Treatment of Cognitive Disorders

Applicant's studies reported herein has led to substantial modifications to previously used methods for gene delivery to entorhinal/hippocampal circuitry to treat cognitive disorders These modifications are based on: 1) primate studies delivering gene therapy vectors to the entorhinal cortex and hippocampus, and 2) expanding clinical experience in 3 current clinical gene therapy trials using MR guidance, Convection Enhanced Delivery (CED) and gadoteridol co-infusion. These considerations take into account new knowledge that the relationship between infusate volume and vector distribution depends on precise needle location in the entorhinal cortex, variable vector dispersion in gray versus white matter, the presence of perivascular spaces (that act as vector sinks during vector infusion), the irregular anatomic architecture of the entorhinal cortex, and the possibility of vector reflux along the infusion tract. These factors require a distinctively higher range of vector volume to effectively fill the target brain structure, specific rates of vector infusion, and specific vector doses (concentrations).

The volume of the human entorhinal cortex is 1500 mm³, both by high resolution brain MR imaging and anatomical measures [Hasan et al., J. Neuroimaging 26 (2015); Fischl et al., Neuroimage 47, (2009); Juottonen et al., Neurobiol. Aging 19, (1998); Bunce et al., J Alzheim Dis 30, (2012)]. The ratio of vector volume of infusion in the brain to volume of distribution (Vi/Vd) ranges from 1:1 to 1:3 in grey matter (ratio of 1:1 means same volume of infusion and volume of distribution, while 1:3 ratio means that the volume of infusion covers 3 times that volume of brain tissue). Thus, without being bound by theory, Applicant calculates that a total vector volume of 500-1500 μl is required to fill the entorhinal cortex. Applicant aimed to fill at least half this volume, requiring infusion of 250-750 μl per hemisphere the brain. Applicant divided this vector volume among 3-4 infusion sites, and cap the maximum infusion volume per hemisphere to 750 μl. Similar to other intracranial gene therapy trials in the brain, adequate coverage will require a range in volume of infusion per site to most effectively fill the irregular architecture of the entorhinal cortex. MRI imaging can be used to track vector distribution and vector distribution in real-time to guide infusion volume in the described range can be assessed.

In monkeys, Applicant infused a volume of 60-435 μl at a vector concentration of 3×10¹¹ vg/ml to achieve 50-100% fill of the entorhinal cortex, as shown in Table 1 below. The volume of the rhesus monkey entorhinal cortex (255 mm³) [5] is ⅙^{th} that of the human entorhinal cortex. Projecting Applicant's preclinical infusion volumes from monkeys to humans to achieve 50% coverage of EC, the corresponding infusion volumes in humans range from 360-2610 μl or alternatively 250-750 μl per hemisphere.

TABLE 1

| | | Total Left | | | Total Right | |
|---|---|---|---|---|---|---|
| | Left Vector | Injx Vol | Left % | Right Vector | Injx Vol | Right % |
| Animal | Conc (vg/ml) | (μl) | Coverage | Conc (vg/ml) | (μl) | Coverage |
| 34557 | 1 × 10¹¹ vg/ml | 66 | 33% | 3 × 10¹¹ vg/ml | 48, 48 | 100% |
| 34143 | 1 × 10¹¹ vg/ml | 64 | 14% | 3 × 10¹¹ vg/ml | 64, 73, 123 | 100% |
| 34164 | 1 × 10¹¹ vg/ml | 95, 82, 74 | 27% | 3 × 10¹¹ vg/ml | 52, 79 | 93% |
| 157-1996 | 3 × 10¹¹ vg/ml | 98, 47, 99 | 100% | 3 × 10¹¹ vg/ml | 166, 199, 70 | 100% |
| 314-1998 | 3 × 10¹¹ vg/ml | 29, 66 | 57% | 3 × 10¹¹ vg/ml | 35, 40 | 53% |
| 369-1996 | 3 × 10¹¹ vg/ml | 23, 20, 30 | 75% | 3 × 10¹¹ vg/ml | 30, 12, 30 | 93% |
| 222-1996 | 3 × 10¹¹ vg/ml | 40, 24 | 57% | 3 × 10¹¹ vg/ml | 20, 40 | 50% |
| V000572 | 3 × 10¹¹ vg/ml | 43, 34 | 54% | 3 × 10¹¹ vg/ml | 49, 34 | 50% |

The volumes Applicant originally cited to infuse in humans (up to 125 μl per site for a maximum of 375 μl per hemisphere) were too conservative. Detailed review of new results from additional monkeys and expanding recent experience in human vector delivery by Applicant resulted in these new guidelines: higher volumes than originally cited are required to achieve adequate filling of target structures in humans.

The revised parameters are based on the study of an additional 25 monkeys that allowed Applicant to develop this specific expertise. It was found that previous doses described in the literature was ineffective (1×10e1), and that a minimum does of 3×10¹¹ vg/ml is the lowest feasible starting dose. To the best of Applicant's knowledge, this information has not been previously disclosed.

Up to 4 infusions are required per hemisphere to effectively treat the human brain. The correct volume of administration in humans includes a range of 360-2610 μl per hemisphere. The literature has described lower vector doses (1×10e10-1×10e11) that Applicant now know are ineffective, and higher doses that applicant now know are too high and will cause toxicity (above 3×10e12). The literature also indicated a method requiring infusion at a range of 15-125 or 15-130 μl/site, meaning that the maximum volume to be infused at each site was 125 μl, for a maximum total per hemisphere of 375 μl.

Extended survival times in monkeys up to 24-30 months identified problems with mis-targeting and seizures using previously published methods. To the best of Applicant's knowledge, this is essential information that is unpublished regarding achieving vector delivery safely. To deliver vector safely, it is essential that delivery is accomplished within the EC region that is limited to its most ventromedial aspect, excluding any vector infusion into the subiculum of the entorhinal cortex and the parasubiculum of the entorhinal cortex, and also excluding direct infusion into the hippocampus. Animals that received infusions that were not within these parameters developed seizures.

Also based on these long-term, unpublished monkey studies, Applicant have identified a vector dose range of 3×10¹¹ to 1×10¹³ vg/ml as the maximum safe and tolerated dose range.

Nagahra et al. (2018) Gene Therapy (2018) 25:104-114 teaches the use of MRI guidance with gadoteridol infusion to reliably infuse AAV2-BDNF into this small brain region. To the best of Applicant's knowledge, the prior art does not provide specific methods for targeting a subportion of the entorhinal cortex (thenventromedial region) and avoiding pre-subiculum, parasubiculum, subiculum and hippocampus, and the literature does not address relay accurate volume considerations and vector concentrations. For example, the literature did not disclose specific details regarding: 1) vector concentrations that are effective for use in humans (for example, to the best of Applicant's knowledge, the published reports of 3×10¹¹ vg/ml but Applicant recommends human dose from about 3×10¹¹ vg/ml, 1×10¹² vg/ml, and up to 1×10¹³ vg/ml. To the best of Applicant's knowledge, the higher doses that applicant studied in monkeys have not been reported or published), 2) effective volumes of infusion for humans.

To the best of Applicant's knowledge, the literature does not identify optimal infusion rate parameters for achieving vector spread within the target region of the brain. The rate of infusion of vector using a continuous infusion pump was determined in non-human primates to fall within a specified range of 1-15 μl/min. This infusion rate achieves adequate vector spread and coverage of the intended target. Lower rates do not spread sufficiently far in the target structure; higher rates leak from the intended target region, constituting safety risks.

The choice of design of infusion needles for gene delivery into the brain should depend on testing of specially designed infusion needles for different regions of the brain. In the entorhinal cortex, Applicant has discovered that a needle “step” design, in which the first several millimeters of the needle are narrow, followed by a subsequent expansion in the diameter of the needle, is an effective means of preventing reflux of the infusion up the needle track. In the entorhinal cortex region the optimal needle design includes a single, widened “step” (expansion in outer diameter) located a distance between 1 and 5 mm from the tip of the infusion needle. This reduces vector reflux and loss up the infusion tract. Inadvertent spread can compromise safety. This knowledge is essential for effective implementation of gene therapy vectors to this brain region.

### Vector Concentrations: Lower Doses are Ineffective and Higher Doses are Toxic: An Ideal Range is Established

The choice of 3×10¹¹ vg/ml as the minimum effective vector titer is based on an earlier Primate Study 1006 (see Table 2), which found that a concentration of 1×10¹¹ vg/ml was insufficient to effectively treat the targeted brain region, the entorhinal cortex. The rostral-to-caudal length of human entorhinal cortex is 30 mm, but vector infused at a titer of 1×10¹¹ vg/ml drove BDNF expression only within 1 mm of the infusion site. This was the case despite the use of convection enhanced delivery (CED) and infusion volumes comparable to those used in monkeys that received a higher vector titer of 3×10¹¹ vg/mL. Applicant thus conclude that a titer of 1×10¹¹ vg/ml is insufficient to treat an adequate volume of the degenerating human entorhinal cortex in AD.

In Primate Study 1006 (see Table 2), Applicant also tested a vector titer of 3×10¹¹ vg/ml. Vector infused at this concentration drove BDNF expression up to 8 mm from the infusion site (mean spread 5.21±0.71 mm per site), using infusion volumes similar to 1×10¹¹ vg/mL infusions. Primate Study 1006 (see Table 2) also found a vector concentration of 3×10¹¹ vg/ml to be safe. Thus, given that this was the lowest vector concentration that effectively delivered BDNF to an adequate volume of entorhinal cortex, this is designated as the minimum effective vector concentration. Previous to this work, it was thought that lower vector doses could be effective. They are not, based on new and extensive primate distribution and dosing studies.

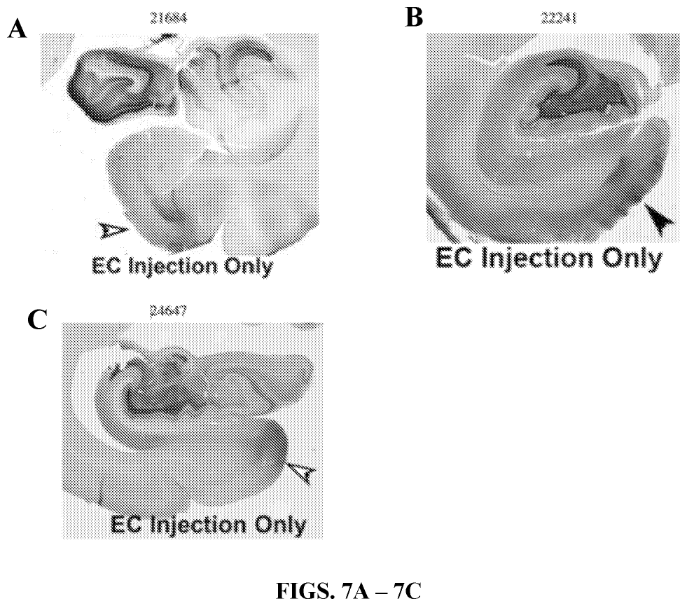

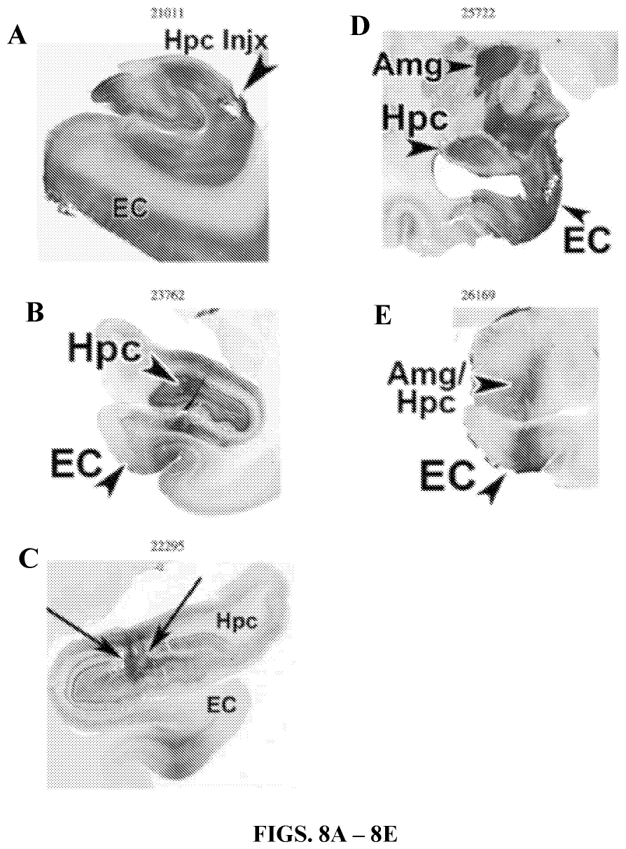

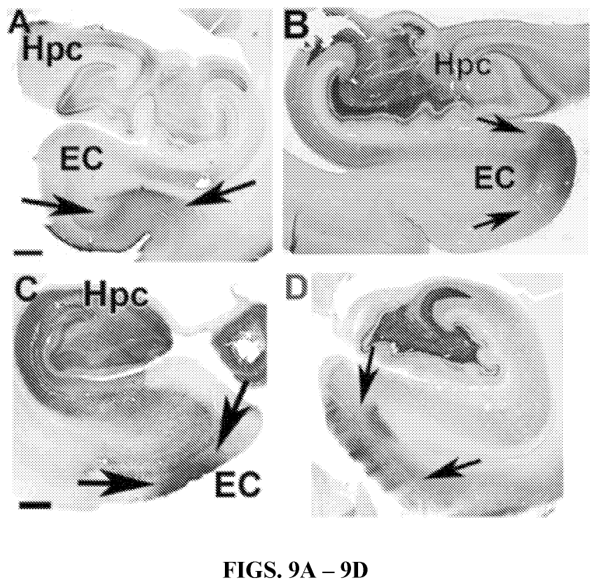

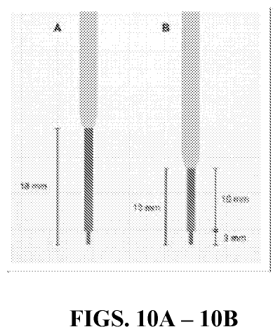

Regarding the higher vector titer of 1×10¹² vg/ml-1×10¹³ vg/ml: Primate Study 1004 (see Table 2) showed that this dose is tolerated when accurately targeted to the entorhinal cortex These injections at titers up to 10¹³ vg/ml resulted in good entorhinal expression without toxicity (seizures) associated with higher doses or vector mistargeting. Arrowheads below indicate regions of BDNF gene expression after vector infusion. See FIG. 7.

This study demonstrates that infusions of AAV2-BDNF in 5 monkeys in regions adjoining the entorhinal cortex—the hippocampus, amygdala, pre-subiculum, parasubiculum and subiculum, resulted in safety problems (seizures). This toxicity was previously unknown and was not anticipated, and highlights the need for precise infusions into the target region of the entorhinal cortex to safely and effectively treat memory disorders.

### A New Range of Vector Volumes are Required to Accurately and Safely Treat Memory Disorders

The volume of the human entorhinal cortex is 1500 mm³, both by high resolution brain MR imaging and anatomical measures [Hasan et al., J. Neuroimaging 26 (2015); Fischl et al., Neuroimage 47, (2009); Juottonen et al., Neurobiol. Aging 19, (1998); Bunce et al., J Alzheim Dis 30, (2012)]. The ratio of vector volume of infusion in the brain to volume of distribution (Vi/Vd) ranges from 1:1 to 1:3 in grey matter (ratio of 1:1 means same volume of infusion and volume of distribution, while 1:3 ratio means that the volume of infusion covers 3 times that volume of brain tissue). Thus, Applicant calculates that a total vector volume of 500-1500 μl is required to fill the entorhinal cortex. Applicant's aim in this trial is to fill at least half this volume, requiring infusion of 250-750 μl per hemisphere the brain. Applicant propose to divide this vector volume among 3-4 infusion sites, and propose capping the maximum infusion volume per hemisphere to 750 μl. Similar to other intracranial gene therapy trials in the brain, adequate coverage will require a range in volume of infusion per site to most effectively fill the irregular architecture of the entorhinal cortex. Moreover, because applicant are using MR imaging to track vector distribution, applicant will be able to accurately assess vector distribution in real-time to guide infusion volume in the described range.

In monkeys, applicant infused a volume of 60-435 μl at a vector concentration of 3×10¹¹ vg/ml to achieve 50-100% fill of the entorhinal cortex, as shown in Table 1, above. The volume of the rhesus monkey entorhinal cortex (255 mm³) [Paxinos et al., The Rhesus Monkey Brain in Stereotaxic Coordinates. (Academic Press, 1989)] is ⅙^{th} that of the human entorhinal cortex. Projecting applicant preclinical infusion volumes from monkeys to humans to achieve 50% coverage of EC, the corresponding infusion volumes in humans would range from 250-2610 μl per hemisphere. These volumes are much higher than originally anticipated and disclosed in the literature; the new parameters have been established by extensive monkey empirical testing and by recent human experience by the inventors. Consideration of the scaling factor from monkey to human has been critical in applicant clinical experience to date, where higher volumes than originally predicted were required to achieve adequate filling of target structures in humans.

### Infusion Rate Must be Precisely Controlled to Accurately and Safely Treat Memory Disorders

Previous methods of delivering AAV into the parenchyma by simple injection have not been effective at distributing vector over significant volumes of target tissue. In contrast, a technique known as CED (convection-enhanced delivery) uses a specially designed reflux resistant cannula to permit high infusion pressures to be exerted at the cannula tip, thereby overcoming the interstitial pressure in the brain and allowing the infusate to permeate large volumes of targeted areas in a homogeneous manner. Moreover, to monitor the distribution of the infusate by magnetic resonance (MR)-imaging, a chelated Gadolinium contrast reagent (gadoteridol) is included in the infusate. The distribution of the gadoteridol closely matches distribution of the infused AAV2 vector with no evidence of toxicity⁷⁻⁹. Pre-clinical and clinical studies have shown that codelivery of gadoteridol, a widely used contrast agent for MR-imaging marketed for intravenous administration and used in this protocol off-label, and real-time MR-imaging helps to ensure that the target region is exposed to the investigational agent while minimizing exposure of non-targeted CNS tissues^{10,11}. The MR images acquired during the CED procedure will be analyzed after surgery to measure the volume of gadoteridol contrast enhancement. Calculations will be made of the total volume of gadoteridol distribution and percentage of entorhinal cortex coverage by gadoteridol. Correlations between coverage and other outcome measures, such as clinical cognitive measures, may be assessed.

In extensive pre-clinical testing in monkeys using infusion parameters ranging from 0.1 μl/min to 15 μl/min, Applicant have identified a new range on infusion rates that support consistent, safe and effective gene delivery to the entorhinal cortex for treating memory disorders: 1-15 μl/min. Lower infusion rates than this result in insufficient spread by MRI whereas higher infusion rates cause local tissue damage. To the best of Applicant's knowledge, this range is novel and inventive from the prior arts and this rate is crucial for practicing this art. See Table 2.

TABLE 2
Pre-Clinical Studies - Summary of AAV2-BDNF Infused into Entorhinal Cortex

| | | | | | | | AAV2-BDNF | | | | | | |
|---|---|---|---|---|---|---|---|---|---|---|---|---|---|
| | | | | | | | Vector | Delivery | | | Total Dose | | |
| | | Age Range | | | | | Product and | Device | | | Volume | | |
| | Animal | (Age at | | Study | | | Concentration | Used in | Dosing | Anatomic | per Brain | Dose Administered | |
| Study Title/Number | Species | Surgery) | | Duration | | N | (Titer vg/ml) | Study | Procedure | Target | (μl) | (Total vg per brain) | |
| Study 2001: | Rat | 3 | Mo | 1 | Mo | 5 | UNC 1 × 10¹² | Stoelting | Stereotaxic | Entorhinal | 5 | 5 × | 10⁹ |
| Biological Activity | | | | | | | | 310 | Procedure | Cortex | | | |
| of BDNF Gene | | | | | | | | Syringe | (No MR | | | | |
| Delivery: AAV2- | | | | | | | | Pump | Guidance) | | | | |
| BDNF Gene | | | | | | | | w/ | | | | | |
| Delivery Prevents | | | | | | | | Hamilton | | | | | |
| Entorhinal Cortical | | | | | | | | Syringe | | | | | |
| Neuronal Death | | | | | | 4 | Control | As | As | Entorhinal | 5 | 0 | |
| After Perforant | | | | | | | | Above | Above | Cortex | | | |
| Path Lesions and | | | | | | | | | | | | | |
| is Equipotent to | | | | | | | | | | | | | |
| Lenti-BDNF | | | | | | | | | | | | | |
| Study 2002: | Rat | 3 | Mo | 1 | Mo | 6 | UNC 1 × 10¹¹ | Stoelting | Stereotaxic | Entorhinal | 5 | 5 × | 10⁸ |
| Biological Activity | | | | | | | | 310 | Procedure | Cortex | | | |
| of Low-Titer | | | | | | | | Syringe | (No MR | | | | |
| AAV2-BDNF: | | | | | | | | Pump | Guidance) | | | | |
| Gene Delivery of | | | | | | | | w/ | | | | | |
| AAV2-BDNF | | | | | | | | Hamilton | | | | | |
| Prevents | | | | | | | | Syringe | | | | | |
| Entorhinal Cortical | | | | | | 8 | UNC 3 × 10¹¹ | As | As | Entorhinal | 5 | 1.5 × | 10⁹ |
| Neuronal Death | | | | | | | | Above | Above | Cortex | | | |
| After Perforant | | | | | | | | | | | | | |
| Path Lesions | | | | | | | | | | | | | |
| | | | | | | 5 | Control | As | As | Entorhinal | 5 | 0 | |
| | | | | | | | | Above | Above | Cortex | | | |
| Study 2003: | Rat | 3 | Mo | 3 d, | | 43 | CHOP (GLP) | Stoelting | Stereotaxic | Entorhinal | 8 | 2.4 × | 10⁹ |
| Safety/Toxicity/ | | | | 28 d, | | | 3 × 10¹¹ | 310 | Procedure | Cortex | | | |
| Biodistribution of | | | | 3 mo, | | | | Syringe | (No MR | | | | |
| BDNF Gene | | | | 6 mo, | | | | Pump | Guidance) | | | | |
| Delivery to the | | | | 12 mo | | | | w/ | | | | | |
| Entorhinal Cortex | | | | | | | | Hamilton | | | | | |
| in Fischer 344 | | | | | | | | Syringe | | | | | |
| Rats | | | | | | | | | | | | | |
| | | | | | | 44 | CHOP (GLP) | As | As | Entorhinal | 80 | 2.4 × | 10¹⁰ |
| | | | | | | | 3 × 10¹¹ | Above | Above | Cortex | | | |
| | | | | | | 43 | CHOP (GLP) | As | As | Entorhinal | 8 | 2.4 × | 10¹⁰ |
| | | | | | | | 3 × 10¹² | Above | Above | Cortex | | | |
| | | | | | | 40 | Control | As | As | Entorhinal | 8, 80 | 0 | |
| | | | | | | | | Above | Above | Cortex | | | |
| Study 1003: | Rhesus | 5-10 | yr | 1 | yr | 4 | UNC 1 × 10¹² | Stoelting | Stereotaxic | Entorhinal | 160 | 1.6 × | 10¹¹ |
| Delivery of | Monkey | | | | | | | 310 | Procedure | Cortex | | | |
| AAV2-BDNF into | | | | | | | | Syringe | (No MR | | | | |
| the Entorhinal | | | | | | | | Pump | Guidance) | | | | |
| Cortices of Adult | | | | | | | | w/ | | | | | |
| Rhesus Monkeys | | | | | | | | Hamilton | | | | | |
| | | | | | | | | Syringe | | | | | |
| Study 1004: | Rhesus | 17-24 | yr | 2-30 | mo | 8 | UNC or | Stoelting | Stereotaxic | Entorhinal | 160 | 1.6 × | 10¹¹ |
| Delivery of | Monkey | | | | | | Virapur | 310 | Procedure | Cortex | | | |
| AAV2-BDNF into | | | | | | | 1 × 10¹² | Syringe | (No MR | | | | |
| the Entorhinal | | | | | | | | Pump | Guidance) | | | | |
| Cortices of Aged | | | | | | | | w/ | | | | | |
| Rhesus Monkeys | | | | | | | | Hamilton | | | | | |
| | | | | | | | | Syringe | | | | | |
| Study 1005: | Rhesus | 7-10 | yr | 1 | mo | 3 | UNC 1 × 10¹² | Medfusion | MRI | Entorhinal | 62-138 | 0.6-1.4 × | 10¹¹ |
| Delivery of | Monkey | | | | | | | 3500 | Guidance | Cortex | | | |
| AAV2-BDNF into | | | | | | | | Syringe | | | | | |
| the Entorhinal | | | | | | | | pump | | | | | |
| Cortices of Adult | | | | | | | | w/ | | | | | |
| Rhesus Monkeys | | | | | | | | Ceramic | | | | | |
| Using MR- | | | | | | | | Cannula | | | | | |
| Guidance, | | | | | | | | and | | | | | |
| Gadolinium Co- | | | | | | | | MRI | | | | | |
| Infusion, and | | | | | | 3-21 yr | 1-12 mo | 16 | UNC | As | As | Entorhinal | |
| Convection- | | | | | | | | | 3 × 10¹¹ | Above | Above | Cortex | |
| Enhanced Vector | | | | | | | | | | | | | |
| Delivery | | | | | | | | | | | | | |
| Study 1006: | Rhesus | 16-22 | yr | 1-24 | mo | 8 | UNC 1 × 10¹¹ | Medfusion | MRI | Entorhinal | 134-382 | 0.3-1.1 × | 10¹¹ |

| | | | | | |
|---|---|---|---|---|---|
| Delivery of | Monkey | or 3 × 10¹¹ | 3500 | Guidance | Cortex |
| AAV2-BDNF Into | | | Syringe | | |
| the Entorhinal | | | pump | | |
| Cortices of Aged | | | w/ | | |
| Rhesus Monkeys | | | Ceramic | | |
| Using MR- | | | Cannula | | |
| Guidance, | | | and | | |
| Gadolinium Co- | | | MRI | | |
| Infusion, and | | | | | |
| Convection- | | | | | |
| Enhanced Vector | | | | | |
| Delivery | | | | | |

An Infusion Cannula with a Step Design Specifically Adapted for the Entorhinal Cortex is Required to Accurately and Safely Treat Memory Disorders

As noted above, CED (convection-enhanced delivery) uses a specially designed reflux resistant cannula to permit high infusion pressures to be exerted at the cannula tip, thereby overcoming the interstitial pressure in the brain and allowing the infusate to permeate large volumes of targeted areas in a homogeneous manner. A step design in infusion catheters has been developed specifically taking into consideration the gray and white matter anatomy of the entorhinal cortex target region. Testing a variety of step designs, applicant found an optimal step distance to be between 0.5-2.0 mm from the infusion tip. This distance is essential to prevent reflux into subcortical white matter and loss of vector in the wrong target. This step distance was not obvious, and was the result of empirical testing in the rhesus monkey brain entorhinal cortex. Previous step designs, as shown below, have been at longer distances to sui targets like the striatum in treating Parkinson's disease. That design, applied to the entorhinal cortex, would be ineffective.

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that the materials, methods, and examples provided here are representative of preferred embodiments, are exemplary, and are not intended as limitations on the scope of the invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

### REFERENCES

1. Kfomy N, Holmes B B, Jiang H, Holtzman D M, Diamond M I. Trans-cellular propagation of Tau aggregation by fibrillar species. J Biol. Chem. 2012; 287:19440-51,
2. Mirbaha H, Holmes B B, Sanders D W, Bieschke I, Diamond M I. Tau trimers are the minimal propagation unit spontaneously internalized to seed intracellular aggregation. J Biol Chem. 2015; 290:14893-903.
3. Braak H, Braak E. Neuropathological stageing of Alzheimer-related changes. Acta Neuropathol. 1991; 82:239-59.
4. Su J H, Deng G, Cotman C W. Transneuronal degeneration in the spread of Alzheimer's disease pathology: immunohistochemical evidence for the transmission of tau hyperphosphorylation. Neu-robiol Dis. 1997; 4:365-75.
5. de Calignon A, Polydoro M, Suarez-Calvet M, William C, Ada-mowicz DH, Kopeikina Kr, et al. Propagation of tau pathology in a model of early Alzheimer's disease. Neuron. 2012; 73:685-97.
6. Gomez-Isla T, Price J L, McKeel D W, Moths J C, Growdon J H, Hyman B T. Profound loss of layer II entorhinal cortex neurons occurs in very mild Alzheimer's disease. J Neurosci. 1996; 16:4491-4500.
7. Kordower J H, Chu Y, Stebbins G T, DeKosky S T, Cochran E J, Bennett D, et al. Loss and atrophy of layer II entorhinal cortex neurons in elderly people with mild cognitive impairment. Ann Neurol. 2001; 49:202-13.
8. Nagahara A H, Tuszynski M H. Potential therapeutic uses of BDNF in neurological and psychiatric disorders. Nat Rev Drug Disoov. 2011; 10:209-19.
9. Connor B, Young D, Yan Q, Faull R L, Synek B, Dragunow M. Brain-derived neurotrophic factor is reduced in Alzheimer's disease. Brain Res Mol Brain Res. 1997; 49:71-81.
10. Narisawa-Saito M, Wakabayashi K, Tsuji S, Takahashi H, Nawa H. Regional specificity of alterations in NGF, BDNF and NT-3 levels in Alzheimer's disease. Neuroreport. 1996; 7:2925-8.
11. Nagahara A H, Mateling M, Kovacs I, Wang L., Eggert S, Rock-enstein E, et al. Early BDNF treatment ameliorates cell loss in the entorhinal cortex of APP transgenic mice. J Neurosci. 2013; 33:15596-602.
12. Nagahara A H, Merrill D A, Coppola G, Tsukada 5, Schroeder B E, Shaked G M, et al. Neuroprotective effects of brain-derived neu-rotrophic factor in rodent and primate models of Alzheimer's disease. Nat Med. 2009; 15:331-7.
13. Poduslo I F, Curran G L. Permeability at the blood-brain and blood-nerve barriers of the neurotrophic factors: NGF, CNTF, NT-3, BDNF. Brain Res Mot Brain Res. 1996; 36:280-6.
14. Tuszynski M H, Yang J H, Barba D, U H S, Bakay R A, Pay M M, et al. Nerve growth factor gene therapy: activation of neuronal responses in Alzheimer disease. JAMA Neurol. 2015; 72:1139-47.
15. Mai J K, Assheuer J, Paxinos G. Atlas of the Human Brain, Academic Press: San Diego, 2004.
16. Juottonen K, 1 Imkso M P, Insausti R, Lehtovirta M, Pitkanen A, Partanen K, et al. Volumes of the entorhinal and perirhinal cortices in Alzheimer's disease. Neurobiol Aging. 1998; 19:15-22.
17. Pennanen C, Kivipelto M, Tuornainen 5, Hartikainen P, Hanninen T, Laakso M P, et al. Hippocampus and entorhinal cortex in mild cognitive impairment and early AD. Neurobiol. Aging. 2004; 25:303-10.
18. Saito R, Bringas J R, Mcknight T R, Wendland M F, Mamot C, Drummond D C, et al. Distribution of liposomes into brain and rat brain tumor models by convection-enhanced delivery monitored with magnetic resonance imaging. Cancer Res. 2004; 64:2572-9.
19. Krauze M T, Mcknight T R, Yamashita Y, Bringas J, Noble C O, Saito R, et al. Real-time visualization and characterization of liposomal delivery into the monkey brain by magnetic resonance imaging. Brain Res Brain Res Protoc. 2005; 16:20-6.
20. Fiandaca M S, Varenika V, Eberling J, Mcknight T, Bringas J, Piviretto P. et al. Real-time MR imaging of adeno-associated viral vector delivery to the primate brain. Neuroimage. 2009; 47 Suppl 2: T27-35.
21. Su X, Kells A P, Aguilar Salegio E A, Richardson R M, Hadaczek P, Beyer J, et al. Real-time MR imaging with Gadoteridol predicts distribution of transgenes after convection-enhanced delivery of AAV2 vectors. Mot Ther. 2010; 18:1490-5.
22. Krauze M T, Forsayeth J, Yin D, Bankiewicz K S. Convection-enhanced delivery of liposomes to primate brain. Methods Enzymol. 2009; 465:349-62.
23. Jahangiri A, Chin A, Flanigan P M, Chen R, Bankiewicz K, Aghi M K. Convection enhanced delivery in glioblastoma a review of pectinical and clinical studies. J Neurosurg. 2017; 126:191-200. In press
24. Richardson R M, Kells A P, Rosenbluth K H, Salegio E A, Fiandaca M S, Larson P S, et al. Interventional MRI-guided putaminal delivery of AAV2-GDNF for a planned clinical trial in Parkinson's disease. Mol Ther. 2011; 19:1048-57.
25. San Sebastian W, Kells A P, Bringas J, Samaranch L, Hadaczek P, Ciesielska A, et al. Safety arid tolerability of MRI-guided infusion of AAV2-hAADC into the mid-brain of non-human primate. Mol Ther Methods Clin Dev. 2014; 3:14049.
26. Richardson R M, Kells A P, Martin A J, Larson P S, Stair P A, Piferi P G, et al. Novel platform for MRI-guided convection-enhanced delivery of therapeutics: preclinical validation in nonhuman primate brain. Stereotact Fund Neurosurg. 39. 2011; 89:141-51.
27. Insausti R, Amaral D G, Cowan W M. The entorhinal cortex of the monkey: II. Cortical afferents. J Comp Neurol. 1987; 264:356-95.
28. Witter M P, Van Hoesen G W, Amaral D G. Topographical orga-nization of the entorhinal projection to the dentate gyrus of the monkey. I Neurosci. 1989; 9:216-28.
29. Witter M P, Amaral D G. Entorhinal cortex of the monkey: V. Projections to the dentate gyrus, hippo-campus, and subicular complex. J Comp Neurol. 1991; 307:437-59.
30. Amaral D G. Emerging principles of intrinsic hippo-campal organization. Curr Opin Neurobiol. 1993; 3125-9.
31. Lavenex P, Amaral D G. Hippocampal-neocortical interaction: a hierarchy of associativity. Hippocampus. 2000; 10:420-30.
32. Kirkby D L, Higgins G A. Characterization of perforant path lesions in rodent models of memory and attention. Eur J Neurosci. 1998; 10:823-38.
33. Myhrer T, Naevdal G A. The temporal-hippocampal region and retention: the role of temporo-entorbinal connections in rats. Scand J Psycho’. 1989; 30:72-80.
34. Paxinos G, Huang X F, Toga A W. The Rhesus monkey brain in stereotaxic coordinates. Academic Press; San Diego, 1989.
35. Salegio E A, Kells A P, Richardson R M, Hadaczek P, Forsayeth 1, Bringas J, et al. Magnetic resonance imaging-guided delivery of adeno-associated virus type 2 to the primate brain for the treatment of lysosomal storage disorders. Hum Gene Ther. 2010; 21:1093-103.
36. Gimenez F, Krauze M T, Valles F, Hadaczek P, Bringas J, Sharma N. et al. Image-guided convection-enhanced delivery of GDNF protein into monkey putamen. Neuroimage. 2011; 54 Suppl 1:5 1 89-95.
37. Liu L, Drouet V, Wu J W, Witter M P, Small S A, Clelland C, et al. Trans-synaptic spread of tau pathology in vivo. PLOS ONE. 2012; 7: El 1302.
38. Takeda S, Wegmann 5, Cho H, DeVos S L, Commins C, Roe A D, et al. Neuronal uptake and propagation of a rare phosphorylated high-molecular-weight tau derived from Alzheimer's disease brain. Nat Commun. 2015; 6:8490.
39. Olsson B, Lautner R, Andreasson U, Ohrfelt A, Portelius E, Bjerke M, et al. CSF and blood biomarkers for the diagnosis of Alzheimer's disease: a systematic review and meta-analysis. Lancet Neurol. 2016; 15:673-84.
40. Dubois B, Hampel H, Feldman H H, Scheltens P, Aisen P, Andrieu 5, et al. Preclinical Alzheimer's disease: definition, natural history, and diagnostic criteria. ALzheimers Dement. 2016; 1/292-323.
41. Bevan A K, Duque 5, Foust K D, Morales P R, Braun L, Schmetzer L, et al. Systemic gene delivery in large species for targeting spinal cord, twain, and peripheral tissues for pediatric disorders. Mol Ther. 2011; 19:1971-80.
42. Arvanitakis Z, Tuszyn ski MH, Bakay R, Arends D, Potkin 5, Bartus R, et al. Interim data from a phase 1 clinical trial of AAV-NGF (CERE-110) gene delivery in Atzheimers disease. AbstrAm Acad Neurol. 2007: p05.071.
43. Rafii M S, Baumann T L, Bakay R A, Ostrove J M, Siffert J, Fleisher A S. et al. A phase’. study of stereotactic gene delivery of AAV2-NGF for Alzheimer's disease. Atzheimers Demerit. 2014; 10:571-81.
44. Fiandaca M S, Forsayeth J R, Dickinson P J, Bankiewicz K S. Image-guided convection-enhanced delivery platform in the treatment of neurological diseases. Neurotherageutics 2008; 5 (1): 123-7.
45. Salegio E A, Bringas J, Bankiewicz K S. MRI-guided delivery of viral vectors. Methods Mol Biol. 2016; 1382:217-30.
46. Amaral D G, Insausti R, Cowan W M. The entorhinal cortex of the monkey: I. Cytoarchitectonic organization. J Comp Neural. 1987; 264:326-55.

1. A method for improving cognitive function in a subject in need thereof comprising administering to a ventromedical nucleus of the subject a polynucleotide encoding brain-derived neurotrophic factor (BDNF) at a dose between about 3×10¹¹ vg/ml to about 1×10¹³ vg/ml administered at an infusion rate between about 0.001 ml/minute to about 0.015 ml/minute and an infusion volume between about 250 μl to about 750 μl per hemisphere, thereby improving cognitive function in the subject.

2. The method of claim 1, wherein the subject suffers from a condition selected from Alzheimer's disease (AD), mild cognitive impairment, pre-symptomatic AD, frontotemporal dementia, or lewy body dementia.

3. The method of claim 1, wherein the subject is a mammal or a human.

4. The method of claim 1, wherein the polynucleotide further comprises an expression vector and the polynucleotide is operatively linked to regulatory nucleotides to drive expression of the polynucleotide.

5. The method of claim 1, wherein the administering is by convection-enhanced delivery (CED).

6. The method of claim 1, wherein the CED comprises of an infusion catheter with a step distance between about 0.5 mm to about 2.0 mm from the infusion tip.

7. The method of claim 1, wherein administering is not to a presubiculum, a parasubiculum, a subiculum or a hippocampus.

8. The method of claim 1, wherein polynucleotide is administered at 3 or 4 infusion sites.

9. The method of claim 1, wherein the dose comprises at least 3×10¹¹ vg/ml.

10. The method of claim 1, wherein administration comprises a dose selected from the group of between about 3×10¹¹ vg/ml to about 5×10¹¹ vg/ml, between about 4×10¹¹ vg/ml to about 6×10¹¹ vg/ml, between about 5×10¹¹ vg/ml to about 7×10¹¹ vg/ml, 6×10¹¹ vg/ml to about 8×10¹¹ vg/ml, between about 7×10¹¹ vg/ml to about 9×10¹¹ vg/ml, between about 8×10¹¹ vg/ml to about 1×10¹² vg/ml, between about 9×10¹¹ vg/ml to about 2×10¹² vg/ml, between about 1×10¹² vg/ml to about 3×10¹² vg/ml, 2×10¹² vg/ml to about 4×10¹² vg/ml, 3×10¹² vg/ml to about 5×10¹² vg/ml, 4×10¹² vg/ml to about 6×10¹² vg/ml, 5×10¹² vg/ml to about 7×10¹² vg/ml, 6×10¹² vg/ml to about 8×10¹² vg/ml, 7×10¹² vg/ml to about 9×10¹² vg/ml, or 8×10¹² vg/ml to about 1×10¹³ vg/ml.

11. The method of claim 1, wherein the expression vector is selected from a plasmid, a liposome, a lentiviral vector, an adenoviral vector, or an adeno-associated vector (AAV).

12. A method for delivering an expression vector to a ventromedial nucleus of a subject in need thereof, comprising infusion of the vector by:
(a) an infusion catheter with a step distance between about 0.5 mm to about 2.0 mm from the infusion tip;
(b) an infusion rate between about 0.001 ml/minute to about 0.015 ml/minute;
(c) an infusion volume between about 250 μl to about 750 μl per hemisphere, wherein the infusion occurs between about 3 to about 4 infusion sites; and
(d) a dose between about 3×10¹¹ vg/ml to about 1×10¹³ vg/ml, and
wherein the delivery avoids one or more of: a presubiculum, a parasubiculum, a subiculum, or a hippocampus regions.

13. The method of claim 12, wherein the subject is suffering from a cognitive disorder.

14. The method of claim 12, wherein the expression vector further comprises a therapeutic polynucleotide.

15. The method of claim 14, wherein the polynucleotide encodes brain-derived neurotrophic factor (BDNF).

16. The method of claim 12, wherein the administration comprises a dose selected from the group of: between about 3×10¹¹ vg/ml to about 5×10¹¹ vg/ml, between about 4×10¹¹ vg/ml to about 6×10¹¹ vg/ml, between about 5×10¹¹ vg/ml to about 7×10¹¹ vg/ml, 6×10¹¹ vg/ml to about 8×10¹¹ vg/ml, between about 7×10¹¹ vg/ml to about 9×10¹¹ vg/ml, between about 8×10¹¹ vg/ml to about 1×10¹² vg/ml, between about 9×10¹¹ vg/ml to about 2×10¹² vg/ml, between about 1×10¹² vg/ml to about 3×10¹² vg/ml, 2×10¹² vg/ml to about 4×10¹² vg/ml, 3×10¹² vg/ml to about 5×10¹² vg/ml, 4×10¹² vg/ml to about 6×10¹² vg/ml, 5×10¹² vg/ml to about 7×10¹² vg/ml, 6×10¹² vg/ml to about 8×10¹² vg/ml, 7×10¹² vg/ml to about 9×10¹² vg/ml, or 8×10¹² vg/ml to about 1×10¹³ vg/ml.
